(19) United States
(12) Patent Application Publication
(10) Pub. No.: US 2019/0357890 A1
(43) Pub. Date: Nov. 28, 2019

(54) ULTRASOUND OBSERVATION DEVICE AND METHOD FOR ULTRASOUND OBSERVATION

(71) Applicant: OLYMPUS CORPORATION, Tokyo (JP)

(72) Inventor: Masataka ITABASHI, Toyko (JP)

(73) Assignee: OLYMPUS CORPORATION, Tokyo (JP)

(21) Appl. No.: 16/537,779

(22) Filed: Aug. 12, 2019

Related U.S. Application Data
(63) Continuation of application No. PCT/JP2018/010811, filed on Mar. 19, 2018.

(30) Foreign Application Priority Data
Mar. 21, 2017 (JP) 2017-054710

(51) Int. Cl. A61B 8/08; A61B 8/00

(52) U.S. Cl. CPC A61B 8/5207; A61B 8/461; A61B 8/0891; A61B 8/469; A61B 8/4483; A61B 8/5238

(57) ABSTRACT

An ultrasound observation device includes a processor including hardware, the processor being configured to: generate an ultrasound image containing a transducer region based on an ultrasound signal received from an ultrasound transducer configured to emit ultrasound to an observation target and receive the ultrasound reflected on the observation target; cause a display to display the ultrasound image; receive a command input to move a region of interest set in the ultrasound image; and move, when a movement vector having a moving direction and a moving amount based on the command input passes through a predetermined region containing at least the transducer region in the ultrasound image, the region of interest to a discontinuous position on an opposite side with the transducer region interposed in the moving direction.

This application is a continuation of PCT International Application No. PCT/JP2018/010811 filed on Mar. 19, 2018, which designates the United States, incorporated herein by reference, and which claims the benefit of priority from Japanese Patent Application No. 2017-054710, filed on Mar. 21, 2017, incorporated herein by reference.

### BACKGROUND

The present disclosure relates to an ultrasound observation device, and a method for ultrasound observation.

Ultrasound may be applied to observe the characteristics of a biological tissue or material of an observation target. Specifically, the ultrasound is emitted to an observation target, and an ultrasound echo reflected by the observation target is subjected to a predetermined signal processing, so that information on the characteristics of the observation target is acquired.

For the observation on the biological tissue in a body using the ultrasound, there is used an ultrasound observation system which includes an ultrasound endoscope equipped with an ultrasound transducer at a distal end of an insertion portion. The ultrasound observation system generates an image in which biological information is added to an ultrasound image in various types of operation modes such as a flow mode, an elastography mode, and a contrast mode for a more detailed observation and for an improved accuracy in comprehensive result from the observation of other aspects. Specifically, a region of interest (ROI) is set on a B-mode reference image, and a process such as calculation corresponding to a predetermined operation mode is performed on the region of interest, so that an image in which the biological information is added to the ultrasound image is generated. When the region of interest is set, a user moves the region of interest using an input device such a track ball or a track pad (for example, see JP 2010-63548 A).

When the region of interest is moved using the track ball or a track pad, the region of interest is rotatably moved about a transducer region (an ultrasound transducer imaged in the ultrasound image) by an operation to the right or left direction. The region of interest is moved in a depth direction by an operation to the upward or downward direction. In addition, when the track pad is used, the region of interest is moved in an operation direction with respect to an operation surface.

Then, when the user sets the region of interest to a desired position, there is generated an image in which the biological information is added to the ultrasound image according to the operation mode.

### SUMMARY

According to one aspect of the present disclosure, there is provided an ultrasound observation device including a processor including hardware, the processor being configured to: generate an ultrasound image containing a transducer region based on an ultrasound signal received from an ultrasound transducer configured to emit ultrasound to an observation target and receive the ultrasound reflected on the observation target; cause a display to display the ultrasound image; receive a command input to move a region of interest set in the ultrasound image; and move, when a movement vector having a moving direction and a moving amount based on the command input passes through a predetermined region containing at least the transducer region in the ultrasound image, the region of interest to a discontinuous position on an opposite side with the transducer region interposed in the moving direction.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating a configuration of an ultrasound observation system which includes an ultrasound observation device according to a first embodiment;

FIG. 2 is a flowchart illustrating an operation of the ultrasound observation device according to the first embodiment;

FIG. 3 is a diagram illustrating an example of an ultrasound image displayed in a display device;

FIG. 4 is a diagram illustrating a state where a user operates a track pad of an input device;

FIG. 5 is a diagram illustrating a state where an operation controller detects a movement vector;

FIG. 6 is a diagram illustrating a state where the operation controller moves a region of interest;

FIG. 7 is a diagram illustrating a state where an operation controller according to Modification 1-1 moves a region of interest;

FIG. 8 is a diagram illustrating a state where an operation controller according to Modification 1-2 moves a region of interest;

FIG. 9 is a diagram illustrating a state where an operation controller according to Modification 1-3 detects a movement vector;

FIG. 10 is a diagram illustrating a state where the operation controller according to Modification 1-3 moves a region of interest;

FIG. 11 is a diagram illustrating a state where the operation controller according to Modification 1-3 detects a movement vector;

FIG. 12 is a diagram illustrating a state where the operation controller according to Modification 1-3 moves a region of interest;

FIG. 13 is a block diagram illustrating a configuration of an ultrasound observation system which includes an ultrasound observation device according to a second embodiment;

FIG. 14 is a flowchart illustrating an operation of the ultrasound observation device according to the second embodiment;

FIG. 15 is a diagram illustrating a state where the operation controller moves a region of interest;

FIG. 16 is a diagram illustrating a state where a user operates a track pad of an input device;

FIG. 17 is a diagram illustrating a state where a user operates a track pad of an input device;

FIG. 18 is a diagram illustrating a state where a user operates a track pad of an input device; and

FIG. 19 is a diagram illustrating a state where a user operates a track pad of an input device.

### DETAILED DESCRIPTION

Hereinbelow, embodiments of an ultrasound observation device, an operation method of the ultrasound observation device, and an operation program of the ultrasound observation device according to the disclosure will be described with reference to the drawings. Further, the disclosure is not limited to these embodiments. In the following embodiments, an ultrasound observation system equipped with an ultrasound endoscope will be described as an example. However, the disclosure may be generally applied to an ultrasound observation device used in the ultrasound observation system such as an external ultrasound observation system and an ultrasound observation system for industrial use.

In addition, the same or corresponding elements in the drawings will be attached with the same symbol appropriately. In addition, the drawings are illustrated in a schematic manner. It should be noted that a dimensional relation between the elements and a ratio of the elements may be different from the actual ones. Even between the drawings, there may be portions having different dimensional relation and ratio.

### First Embodiment

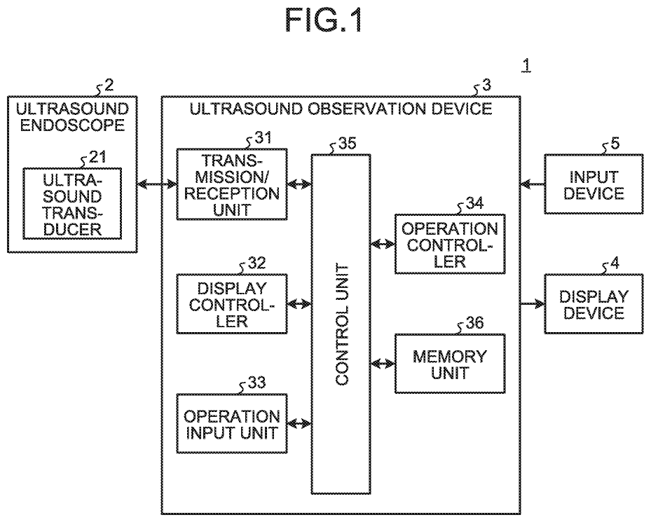

FIG. 1 is a block diagram illustrating a configuration of an ultrasound observation system which includes an ultrasound observation device according to a first embodiment. As illustrated in FIG. 1, an ultrasound observation system 1 includes an ultrasound endoscope 2 which emits an ultrasound toward a subject of an observation target to receive the ultrasound reflected on the subject, an ultrasound observation device 3 which generates an ultrasound image based on an ultrasound signal acquired by the ultrasound endoscope 2, a display device 4 which displays the ultrasound image generated by the ultrasound observation device 3, and an input device 5 which receives an input of a command signal such as setting of an observation mode and setting of an observation condition with respect to the ultrasound observation device 3. The ultrasound observation system 1 can observe the subject in various types of operation modes such as a flow mode, an elastography mode, and a contrast mode.

The ultrasound endoscope 2 includes an ultrasound transducer 21 in its distal end portion. The ultrasound transducer 21 converts an electrical pulse signal received from the ultrasound observation device 3 into an ultrasound pulse (acoustic pulse) to irradiate the subject, and expresses an ultrasound echo reflected on the subject into a voltage change and outputs the echo. The ultrasound transducer 21 is realized by a radial transducer. The ultrasound endoscope 2 may be configured to mechanically scan the ultrasound transducer 21, or may be configured such that a plurality of elements are provided in an array shape as the ultrasound transducer 21, electronically switches the elements related to transmit or receive, and delays the transmitting and receiving of the element to electronically perform scanning.

The ultrasound endoscope 2 typically includes an imaging unit which includes an imaging optical system and an imaging element, and is inserted into an alimentary tract (esophagus, stomach, duodenum, and large intestine) or a respiratory system (trachea and bronchi) of the subject to capture the alimentary tract, the respiratory system, and ambient organs thereof (pancreas, cholecystis, ductus biliferi, biliary tract, lymph gland, mediastinal organ, and blood vessel). In addition, the ultrasound endoscope 2 includes a light guide which guides illumination light which is emitted toward the subject at the time of capturing. The light guide is configured such that a distal end portion reaches the distal end of the insertion portion into the subject of the ultrasound endoscope 2, and a base end portion is connected to a light source device which generates illumination light.

The ultrasound observation device 3 includes a transmission/reception unit 31, a display controller 32, an operation input unit 33, an operation controller 34, a control unit 35, and a memory unit 36.

The transmission/reception unit 31 transmits and receives an electric signal between the imaging unit and the ultrasound transducer 21. The transmission/reception unit 31 is electrically connected to the imaging unit, transmits image information such as an imaging timing to the imaging unit, and receives an imaging signal generated by the imaging unit. In addition, the transmission/reception unit 31 is electrically connected to the ultrasound transducer 21, transmits the electrical pulse signal to the ultrasound transducer 21, and receives an echo signal which is an electric signal received from the ultrasound transducer 21. Specifically, the transmission/reception unit 31 generates the electrical pulse signal based on a predetermined waveform and a transmission timing, and transmits the generated pulse signal to the ultrasound transducer 21.

The transmission/reception unit 31 performs a sensitivity time control (STC) correction to amplify the echo signal with a higher gain as a reception depth is high. The transmission/reception unit 31 performs processing such as filtering on the amplified echo signal, performs A/D conversion, and generates a digital radio frequency signal (RF signal) of a time domain and outputs the signal.

The display controller 32 generates endoscope image data based on the imaging signal, and ultrasound image data corresponding to the electrical echo signal. Further, the display controller 32 overlaps various types of information with the endoscope image data and the ultrasound image data, outputs the data, and controls the displaying of the display device 4. The ultrasound image generated by the display controller 32 is an image containing a transducer region which is the ultrasound transducer 21 reflected on the ultrasound image. Specifically, the ultrasound image is an annular image in which all around periphery (360°) of the ultrasound transducer 21 using the radial ultrasound transducer 21 is captured, and an image in which the periphery (180° or more) of the ultrasound transducer 21 using the radial or convex ultrasound transducer 21 is captured.

The display controller 32 sets a region of interest in the ultrasound image according to a user's command input. Further, the display controller 32 generates an image obtained by overlapping the region of interest on the ultrasound image with a solid line, for example. In addition, the display controller 32 generates an image obtained by adding biological information in the region of interest to the ultrasound image according to the operation mode. For example, the image added with the biological information is generated by adding color using an average value of the biological information in the set region of interest as a reference value. Further, the biological information is bloodstream information in an organ or information of rigidity of a biological tissue, for example. The display controller 32 is realized using a central processing unit (CPU) having calculation and control functions, and various types of calculation circuits.

The operation input unit 33 receives the command signal which is input by the input device 5, and receives various types of information according to the received command signal. As the command signal, there are a command input to move the region of interest set in the ultrasound image, setting of the observation mode and setting of the observation condition (for example, switching of gain and display range, scroll command information (a slide direction and a slide amount of B mode image)), and rotation command information (a rotation direction and a rotation amount of the ultrasound image). For example, the operation input unit 33 receives a command input to associate an operation surface of the input device 5 of a track pad with the display device 4. Specifically, the operation input unit 33 receives a command input such that a moving amount of the command input for the operation surface of the input device 5 and a moving amount of the region of interest in the display device 4 are in a predetermined correlation (for example, the moving amount to the operation surface and the moving amount to the screen of the display device correspond to each other at a ratio of 1:1). The operation input unit 33 is realized using a CPU having calculation and control functions, and various types of calculation circuits.

The operation controller 34 moves the region of interest according to the moving direction and the moving amount based on the command input received by the operation input unit 33. Specifically, the operation controller 34 moves the region of interest to a discontinuous position on an opposite side with a transducer region interposing in the moving direction when the moving direction and the moving amount based on the command input pass through a predetermined region containing at least the transducer region in the ultrasound image. On the other hand, the operation controller 34 continuously moves the region of interest when the moving direction and the moving amount based on the command input do not pass through a predetermined region. The predetermined region is, for example, the transducer region. The operation controller 34 is realized using a CPU having calculation and control functions, and various types of calculation circuits.

The control unit 35 controls the entire ultrasound observation system 1. The control unit 35 is realized using a CPU having calculation and control functions, and various types of calculation circuits. The control unit 35 reads from the memory unit 36 the information memorized and stored by the memory unit 36 and performs various types of calculation processes related to the operation method of the ultrasound observation device 3, thereby integrally controlling the ultrasound observation device 3. Further, the control unit 35 may be configured by using the CPU and the like common to the display controller 32, the operation input unit 33, and the operation controller 34.

The memory unit 36 stores various types of programs to operate the ultrasound observation system 1, and data containing various types of parameters required for the operation of the ultrasound observation system 1. For example, the memory unit 36 stores an initial position (acoustic ray number) of a writing position of the ultrasound image (a transmission start position of the pulse signal).

In addition, the memory unit 36 stores various types of programs containing the operation program to execute the operation method of the ultrasound observation system 1. The operation program may be widely distributed in a form of a computer-readable recording medium such as a hard disk, a flash memory, a CD-ROM, a DVD-ROM, a flexible disk. Further, the various types of programs may be acquired by being downloaded through a communication network. The communication network herein is realized by an existing public line, a local area network (LAN), or a wide area network (WAN), and is realized in a wired or wireless manner.

The memory unit 36 thus configured is realized using a read only memory (ROM) where the various types of programs are installed in advance, and a random access memory (RAM) where calculation parameters, data, and the like of each process are stored.

The display device 4 is connected to the ultrasound observation device 3. The display device 4 is configured using a display panel made of liquid crystal or organic Electro Luminescence (EL). The display device 4 displays the ultrasound image output by the ultrasound observation device 3 and various types of information related to the operation.

The input device 5 includes a track pad which detects movement of a contact position of a contact object touched by the user, such as a user's finger. The input device 5 is electrically connected to the ultrasound observation device 3 through a cable, and outputs a signal of the command input for the track pad to the operation input unit 33.

When the contact object such as an operator's finger comes into contact with the track pad, the input device 5 detects a contact position using a contact sensor and outputs the position to the ultrasound observation device 3. In addition, when the contact object moves while being in contact with the track pad, the moving direction and the moving amount are detected and output to the ultrasound observation device 3. The operation input unit 33 performs a signal processing according to the input contact position, and the moving direction and the moving amount of the contact position based on the received information. Then, the operation input unit 33 calculates a movement vector from the information of movement of the contact position input from the track pad of the input device 5. Further, the input device 5 may include a touch panel in which the contact sensor is overlapped with the display device instead of the track pad.

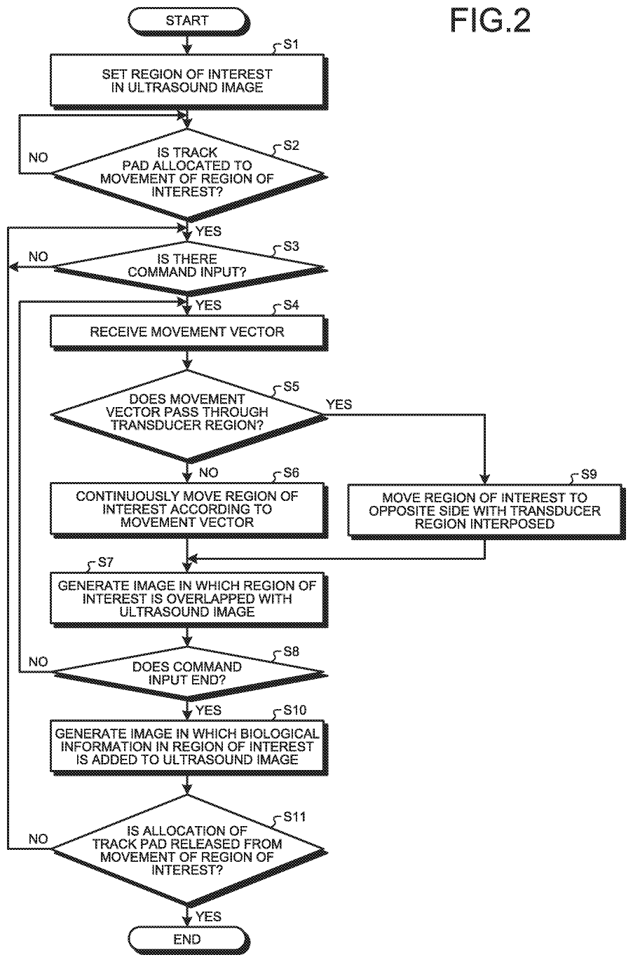

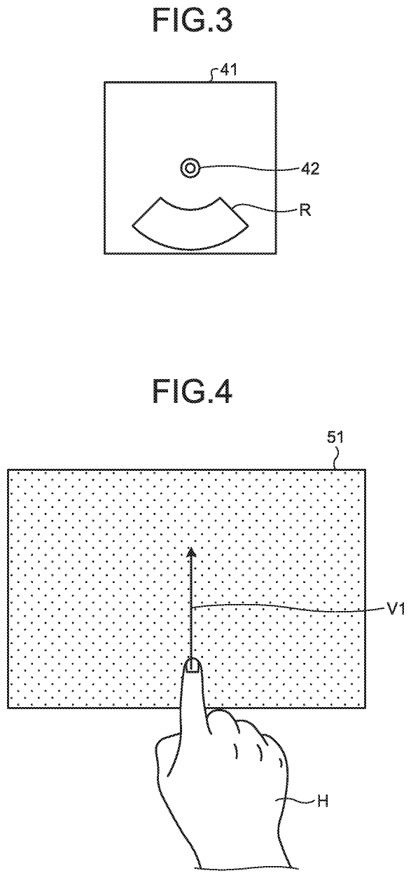

FIG. 2 is a flowchart illustrating an operation of the ultrasound observation device according to the first embodiment. As illustrated in FIG. 2, first, the display controller 32 sets the region of interest in the ultrasound image according to the command input from the input device 5 (Step S1). FIG. 3 is a diagram illustrating an example of an ultrasound image displayed in a display device. As illustrated in FIG. 3, the display controller 32 sets a region of interest R below a transducer region 42 in an ultrasound image 41 according to the command input from the input device 5. For example, the region of interest R has a shape obtained by concentrically dividing a fan shape radially expanding from the center of the transducer region 42 in a depth direction.

Subsequently, the control unit 35 determines whether the track pad of the input device 5 is allocated to the movement of the region of interest R (Step S2). When the control unit 35 determines that the track pad of the input device 5 is not allocated to the movement of the region of interest R (Step S2: No), the determination of Step S2 is repeatedly performed.

On the other hand, when the control unit 35 determines that the track pad of the input device 5 is allocated to the movement of the region of interest R (Step S2: Yes), the control unit 35 determines whether there is a command input to move the region of interest R to the track pad (Step S3). When the control unit 35 determines that there is no command input to move the region of interest R to the track pad (Step S3: No), the determination of Step S3 is repeatedly performed, and the process is on standby until there is a command input of the movement of the region of interest R.

On the other hand, when the control unit 35 determines that there is a command input to move the region of interest R to the track pad (Step S3: Yes), the operation input unit 33 receives the movement vector having the moving direction and the moving amount according to the command input of the user which is detected by the input device 5 (Step S4). FIG. 4 is a diagram illustrating a state where a user operates a track pad of an input device. As illustrated in FIG. 4, when a finger of a user's hand H comes into contact with a track pad 51 and moves upward, the operation input unit 33 detects a movement vector V1 which is directed upward. Further, the movement vector V1 schematically represents a set of minute movement vectors which are repeatedly detected at every minute unit time. The operation input unit 33 may associate the contact position of the track pad 51 to the center of the region of interest R, or may associate the contact position to four corners of the region of interest R. In addition, when there is a command input, the operation input unit 33 may display the contact position to the track pad 51 to be overlapped with the ultrasound image 41, or may highlight the transducer region 42 to be overlapped with the ultrasound image 41.

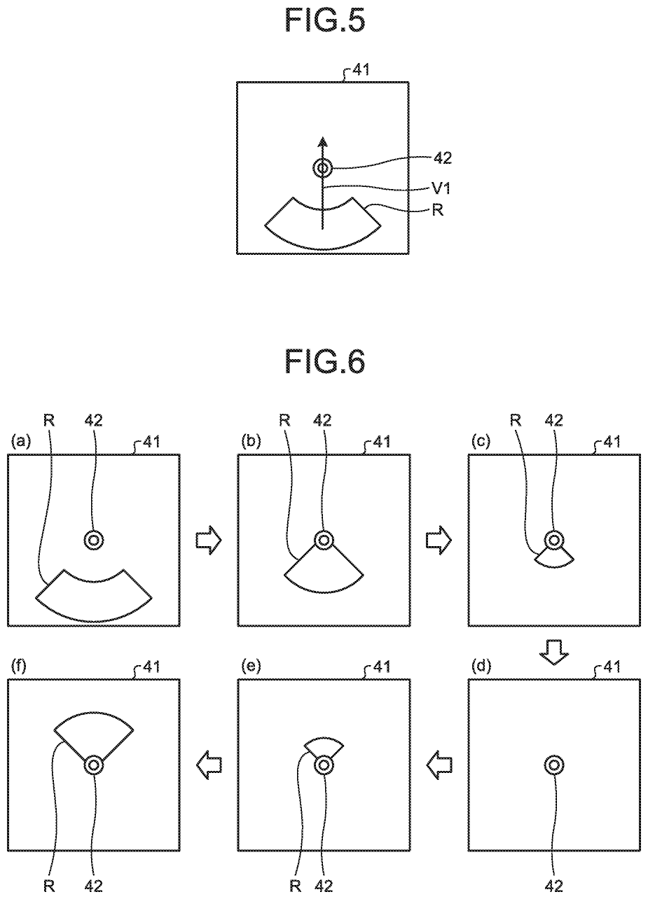

Subsequently, the operation controller 34 determines whether the movement vector received by the operation input unit 33 passes through the transducer region 42 in the ultrasound image 41 (Step S5). FIG. 5 is a diagram illustrating a state where an operation controller detects a movement vector. As illustrated in FIG. 5, the operation controller 34 compares the movement vector V1 with the ultrasound image 41, and determines whether the movement vector passes through the transducer region 42 in the ultrasound image 41.

When the operation controller 34 determines that the movement vector received by the operation input unit 33 does not pass through the transducer region 42 in the ultrasound image 41 (Step S5: No), the operation controller 34 continuously moves the region of interest R according to the movement vector (Step S6). FIG. 6 is a diagram illustrating a state where the operation controller moves a region of interest. As illustrated in (a) to (c) of FIG. 6 or (e) and (f) of FIG. 6, when the operation controller 34 determines that the movement vector (a minute movement vector detected at every minute unit time) received by the operation input unit 33 does not pass through the transducer region 42 in the ultrasound image 41, the operation controller 34 continuously moves the region of interest R upward according to the movement vector. Further, in the first embodiment, when the region of interest R abuts on the transducer region 42, the operation controller 34 gradually reduces the region of interest R according to the movement vector (see (c) of FIG. 6). In addition, the operation controller 34 gradually increases the region of interest R moved to the opposite side with the transducer region 42 interposed up to the original size (see (e) of FIG. 6). When the region of interest R abuts on the transducer region 42, the display controller 32 may highlight the transducer region 42. Then, the display controller 32 generates an image to which the region of interest R moved to the ultrasound image 41 is overlapped with a solid line, for example (Step S7).

Thereafter, the control unit 35 determines whether the command input to the track pad 51 ends (Step S8). When the control unit 35 determines that the command input to the track pad 51 does not end (Step S8: No), the process returns to Step S4 and continues.

In Step S5, when the operation controller 34 determines that the movement vector received by the operation input unit 33 passes through the transducer region 42 in the ultrasound image 41 (Step S5: Yes), the operation controller 34 moves the region of interest R to a discontinuous position on the opposite side with the transducer region 42 interposed in the moving direction as illustrated in (c) to (e) of FIG. 6 (Step S9). Further, in the first embodiment, the operation controller 34 moves the region of interest R to a discontinuous position on the opposite side with the transducer region 42 interposed after a state that the region of interest R temporarily disappears (see (d) of FIG. 6). Then, the display controller 32 generates an image to which the region of interest R moved to the ultrasound image 41 is overlapped with a solid line, for example (Step S7). By displaying the image generated by the display controller 32 is displayed in the display device 4, the user can observe a state that the region of interest R is moving, as illustrated in FIG. 6.

In Step S8, when the control unit 35 determines that the command input to the track pad 51 ends (Step S8: Yes), the display controller 32 generates an image added with the biological information in the region of interest R moved to the ultrasound image 41 according to the operation mode (Step S10).

Thereafter, the control unit 35 determines whether the allocation of the track pad 51 of the input device 5 is released from the movement of the region of interest R (Step S11). When the control unit 35 determines that the allocation of the track pad of the input device 5 is not released from the movement of the region of interest R (Step S11: No), the process returns to Step S3 and continues.

On the other hand, when the control unit 35 determines that the allocation of the track pad 51 of the input device 5 is released from the movement of the region of interest R (Step S11: Yes), a series of processes ends.

As described above, according to the first embodiment, the region of interest R moves to the opposite side with the transducer region 42 interposed according to the user's operation. Therefore, the region of interest R can be moved intuitively with respect to the ultrasound image 41 containing the transducer region 42.

Modification 1-1

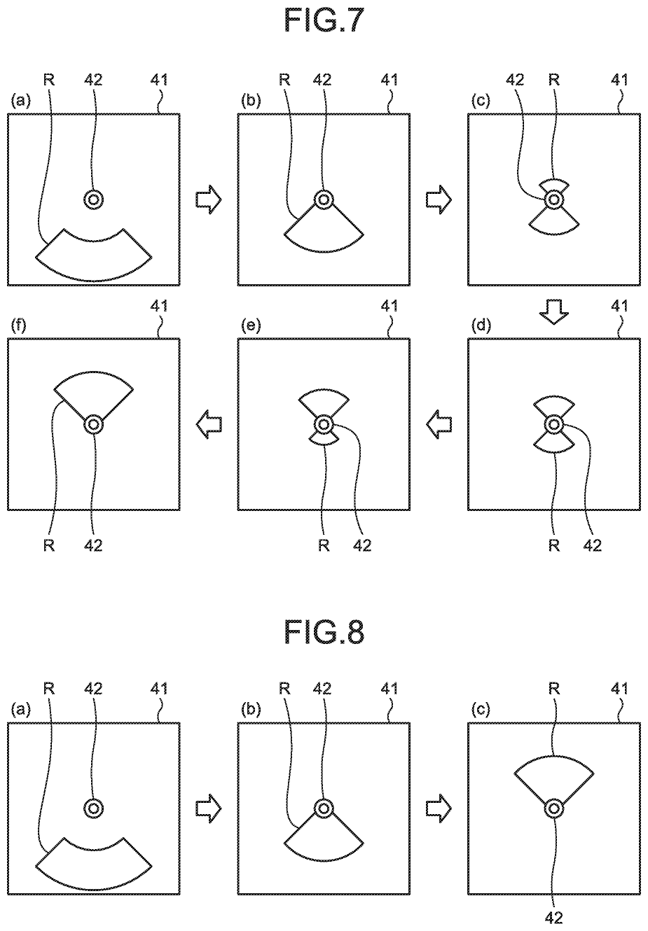

FIG. 7 is a diagram illustrating a state where the operation controller according to Modification 1-1 moves a region of interest. As illustrated in (a) to (f) of FIG. 7, when the operation controller 34 moves the region of interest R to a discontinuous position on the opposite side with the transducer region 42 interposed in the moving direction (Step S9), and if the region of interest R abuts on the transducer region 42, the region of interest R is reduced in the depth direction, and the region of interest R corresponding to the reduced amount may be formed on the opposite side with the transducer region 42 interposed.

Modification 1-2

FIG. 8 is a diagram illustrating a state where the operation controller according to Modification 1-2 moves a region of interest. As illustrated in (a) to (c) of FIG. 8, when the operation controller 34 moves the region of interest R to a discontinuous position on the opposite side with the transducer region 42 interposed in the moving direction (Step S9), and if the region of interest R abuts on the transducer region 42, the entire region of interest R may be moved to the opposite side with the transducer region 42 interposed at a time.

Modification 1-3

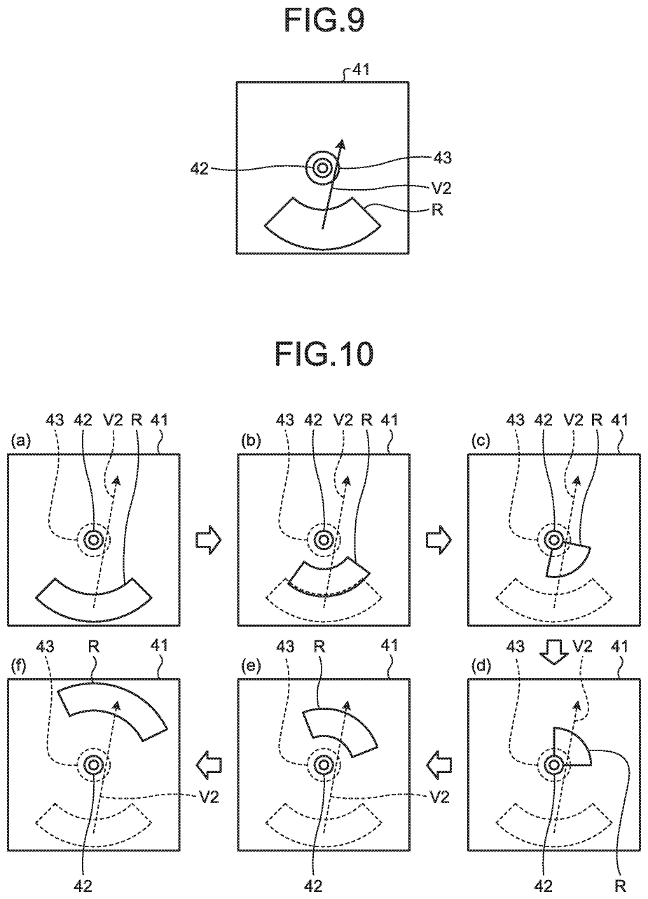

FIG. 9 is a diagram illustrating a state where an operation controller according to Modification 1-3 detects a movement vector. As illustrated in FIG. 9, when a movement vector V2 passes through a region 43 containing the transducer region 42 in the ultrasound image 41, the operation controller 34 moves the region of interest R to a discontinuous position on the opposite side with the transducer region 42 interposed in the moving direction.

FIG. 10 is a diagram illustrating a state where the operation controller according to Modification 1-3 moves a region of interest. As illustrated in (a) to (c) of FIG. 10 or (d) to (f) of FIG. 10, when the operation controller 34 determines that the movement vector (a minute movement vector detected at every minute unit time) received by the operation input unit 33 does not pass through the transducer region 42 in the ultrasound image 41, the operation controller 34 continuously moves the region of interest R along the movement vector. On the other hand, when the operation controller 34 determines that the movement vector received by the operation input unit 33 passes through the transducer region 42 in the ultrasound image 41, the operation controller 34 moves the region of interest R to a discontinuous position on the opposite side with the transducer region 42 interposed along the movement vector as illustrated in (c) to (d) of FIG. 10. More specifically, the region of interest R is moved to a position symmetrical about a straight line which is perpendicular to the movement vector and passes through the center of the transducer region 42. In this way, the region of interest R may be not necessarily moved to a position symmetrical about a point, that is, the center of the transducer region 42.

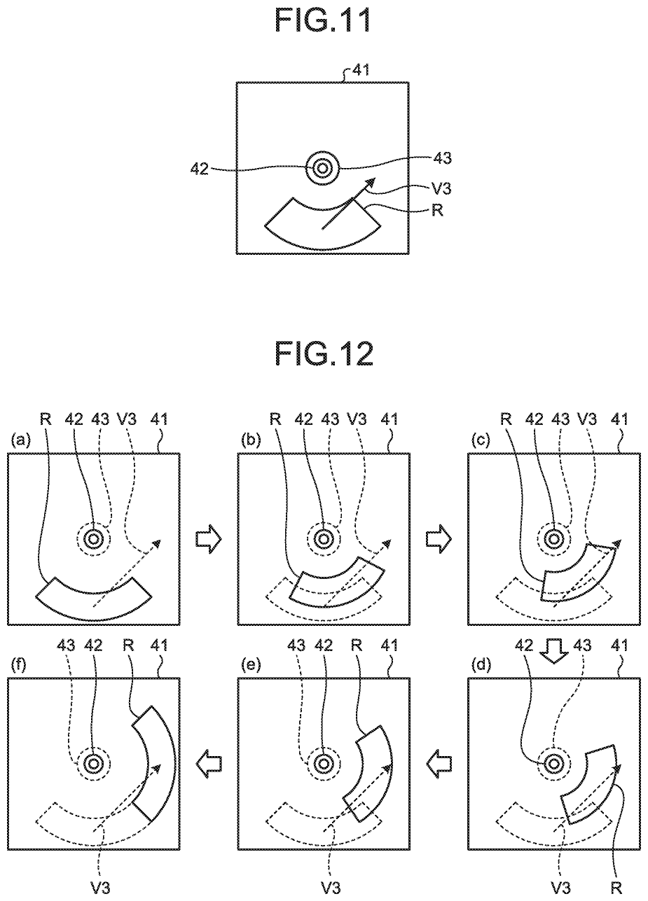

FIG. 11 is a diagram illustrating a state where an operation controller according to Modification 1-3 detects a movement vector. FIG. 12 is a diagram illustrating a state where the operation controller according to Modification 1-3 moves a region of interest. As illustrated in FIG. 11 and (a) to (f) of FIG. 12, when a movement vector V3 does not pass through the region 43 containing the transducer region 42 in the ultrasound image 41, the operation controller 34 continuously moves the region of interest R along the movement vector as illustrated in FIG. 11.

As described above, when the movement vector passes through the region 43 containing the transducer region 42 in the ultrasound image 41, the region of interest R may be moved to a discontinuous position on the opposite side with the transducer region 42 interposed in the moving direction according to the movement vector. In addition, when the movement vector passes through a position deviated from the center of the transducer region 42, the region of interest R may be moved to a discontinuous position on the opposite side with the transducer region 42 interposed along the movement vector in the moving direction.

### Second Embodiment

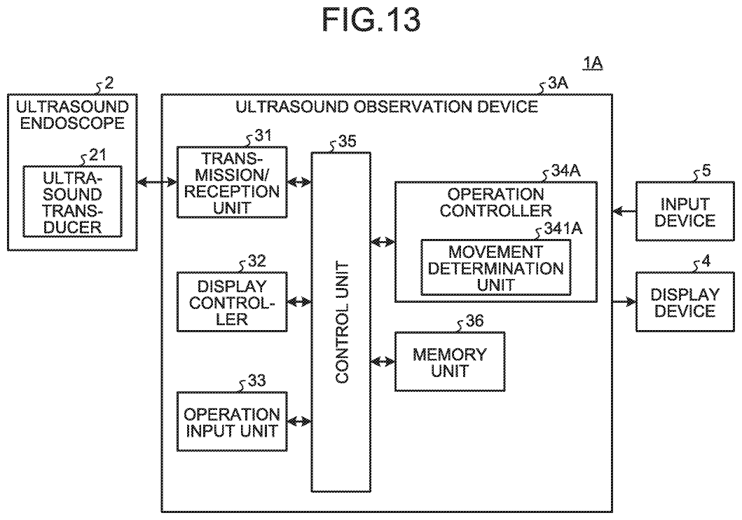

FIG. 13 is a block diagram illustrating a configuration of an ultrasound observation system which includes an ultrasound observation device according to a second embodiment. As illustrated in FIG. 13, an operation controller 34A of an ultrasound observation device 3A in an ultrasound observation system 1A according to the second embodiment includes a movement determination unit 341A. The other configurations are similar to that of the first embodiment, and thus the description will be omitted appropriately.

When a predetermined condition is satisfied, the movement determination unit 341A determines whether the region of interest is moved to a discontinuous position on the opposite side with the transducer region interposed in the moving direction. For example, the movement determination unit 341A determines whether the region of interest is moved to a discontinuous position on the opposite side with the transducer region interposed in the moving direction using at least one of a continuation time of the command input to the operation input unit 33 and the number of times of the command input to the operation input unit 33 after the region of interest abuts on a predetermined region, a moving speed of the command input to the operation input unit 33, and a type of the command input to the operation input unit 33. Specifically, the movement determination unit 341A determines that the region of interest is moved to a discontinuous position on the opposite side with the transducer region interposed in the moving direction when the continuation time of the command input to the operation input unit 33 elapses by a predetermined time or more and when the number of times of the command input to the operation input unit 33 is a predetermined number of times or more after the region of interest abuts on the transducer region, when the moving speed of the command input to the operation input unit 33 is a predetermined speed or more, or when the type of the command input to the operation input unit 33 is an operation using two fingers.

The operation controller 34A moves the region of interest to a discontinuous position on the opposite side with the transducer region interposed in the moving direction according to the determination result of the movement determination unit 341A.

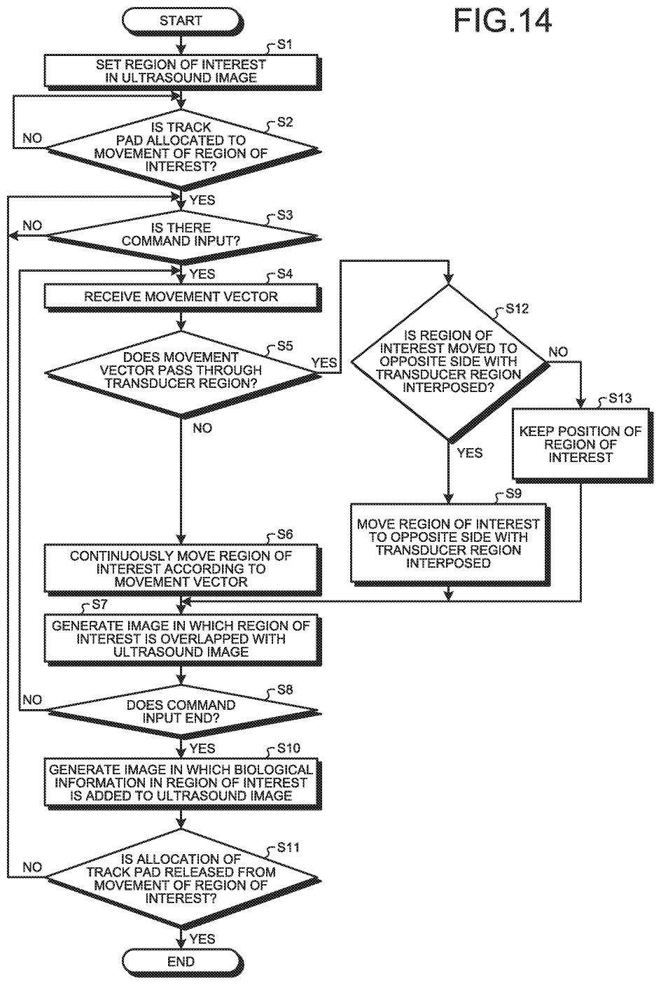

FIG. 14 is a flowchart illustrating an operation of the ultrasound observation device according to the second embodiment. As illustrated in FIG. 14, in Step S5, when the operation controller 34A determines that the movement vector received by the operation input unit 33 passes through the transducer region in the ultrasound image 41 (Step S5: Yes), the movement determination unit 341A determines whether the region of interest is moved to a discontinuous position on the opposite side with the transducer region interposed (Step S12).

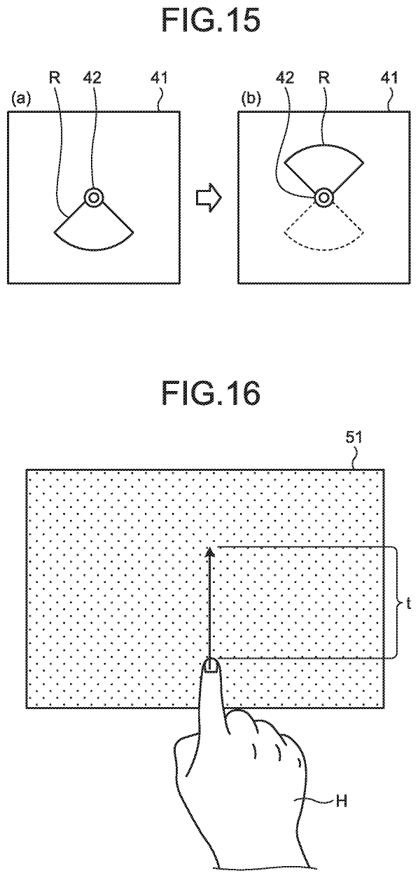

When the movement determination unit 341A determines that the region of interest is moved to a discontinuous position on the opposite side with the transducer region interposed (Step S12: Yes), the operation controller 34A moves the region of interest to the discontinuous position on the opposite side with the transducer region interposed in the moving direction according to the movement vector (Step S9). Thereafter, the process proceeds to Step S7. FIG. 15 is a diagram illustrating a state where the operation controller moves a region of interest. As illustrated in FIG. 15, the operation controller 34A moves the entire region of interest R to a discontinuous position on the opposite side with the transducer region 42 interposed in the moving direction.

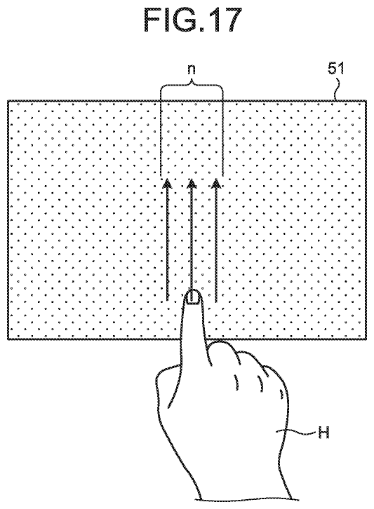

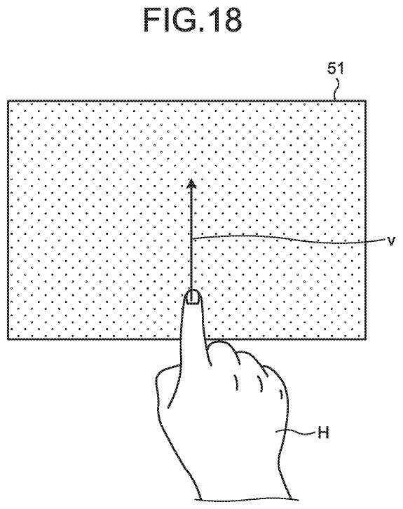

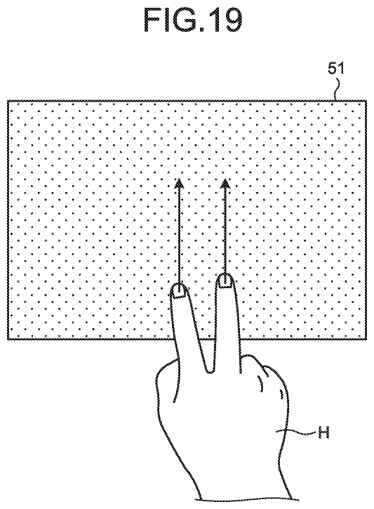

The determination in the movement determination unit 341A will be described specifically. FIGS. 16 to 19 are diagrams illustrating states where a user operates a track pad of an input device. As illustrated in FIG. 16, when a continuation time t of the command input to the operation input unit 33 elapses by a predetermined time or more after the region of interest R abuts on the transducer region 42, the movement determination unit 341A determines that the region of interest R is moved to a discontinuous position on the opposite side with the transducer region 42 interposed in the moving direction. In addition, as illustrated in FIG. 17, when the number n of times of the command input to the operation input unit 33 is a predetermined number of times or more after the region of interest R abuts on the transducer region 42, the movement determination unit 341A determines that the region of interest R is moved to a discontinuous position on the opposite side with the transducer region 42 interposed in the moving direction. In addition, as illustrated in FIG. 18, when the moving speed v of the command input to the operation input unit 33 is a predetermined speed or more, the movement determination unit 341A determines that the region of interest R is moved to a discontinuous position on the opposite side with the transducer region 42 interposed in the moving direction. In addition, as illustrated in FIG. 19, when the type of the command input to the operation input unit 33 is an operation using two fingers, the movement determination unit 341A determines that the region of interest R is moved to a discontinuous position on the opposite side with the transducer region 42 interposed in the moving direction.

On the other hand, when the movement determination unit 341A determines that the region of interest is not moved to a discontinuous position on the opposite side with the transducer region 42 interposed (Step S12: No), the operation controller 34A keeps the position of the region of interest R without moving (Step S13). Thereafter, the process proceeds to Step S7.

As described above, according to the second embodiment, when a predetermined condition is satisfied, the transducer region 42 moves to the opposite side with the transducer region 42 interposed according to the user's operation. Therefore, the region of interest R can be moved intuitively with respect to the ultrasound image 41 containing the transducer region 42.

Further, in the above embodiments, the region of interest R has been described to have a shape obtained by concentrically dividing a fan shape radially expanding from the center of the transducer region 42 in the depth direction, but the disclosure is not limited thereto. For example, the region of interest R may be formed in a circular or polygonal shape.

In addition, in the above-described embodiments, the ultrasound observation device 3 has been described to move the region of interest R based on the command input from the track pad 51 of the input device 5, but the disclosure is not limited thereto. For example, the ultrasound observation device 3 may be configured to move the region of interest R based on the command input from a track ball or a mouse of the input device 5.

According to the disclosure, it is possible to realize an ultrasound observation device, an operation method of the ultrasound observation device, and an operation program of the ultrasound observation device with which a region of interest can be moved intuitively with respect to an ultrasound image containing a transducer region.

The above and other features, advantages and technical and industrial significance of this disclosure will be better understood by reading the following detailed description of presently preferred embodiments of the disclosure, when considered in connection with the accompanying drawings.

What is claimed is:

1. An ultrasound observation device comprising
a processor comprising hardware, the processor being configured to:
generate an ultrasound image containing a transducer region based on an ultrasound signal received from an ultrasound transducer configured to emit ultrasound to an observation target and receive the ultrasound reflected on the observation target;
cause a display to display the ultrasound image;
receive a command input to move a region of interest set in the ultrasound image; and
move, when a movement vector having a moving direction and a moving amount based on the command input passes through a predetermined region containing at least the transducer region in the ultrasound image, the region of interest to a discontinuous position on an opposite side with the transducer region interposed in the moving direction.

2. The ultrasound observation device according to claim 1, wherein the predetermined region is the transducer region.

3. The ultrasound observation device according to claim 1, wherein the processor receives the command input from a track pad configured to detect a contact position of a contact object brought into contact by a user.

4. The ultrasound observation device according to claim 1, wherein the processor continuously moves the region of interest when the movement vector does not pass through the predetermined region.

5. The ultrasound observation device according to claim 1, wherein the processor moves the region of interest to a discontinuous position on an opposite side with the transducer region interposed in the moving direction when the region of interest abuts on the transducer region.

6. The ultrasound observation device according to claim 1, wherein the processor is further configured to determine whether the region of interest is moved to the discontinuous position on the opposite side with the transducer region interposed in the moving direction using at least one of a continuation time of the command input and number of times of the command input after the region of interest abuts on the predetermined region, a moving speed of the command input, and a type of the command input.

7. The ultrasound observation device according to claim 6, wherein the processor moves the region of interest to the discontinuous position on the opposite side with the transducer region interposed in the moving direction according to a result of the determination.

8. A method for ultrasound observation, the method comprising:
generating an ultrasound image containing a transducer region based on an ultrasound signal received from an ultrasound transducer configured to emit ultrasound to an observation target and receive the ultrasound reflected on the observation target;
displaying the ultrasound image;
receiving a command input to move a region of interest set in the ultrasound image; and
moving, when a movement vector having a moving direction and a moving amount based on the command input passes through a predetermined region containing at least the transducer region in the ultrasound image, the region of interest to a discontinuous position on an opposite side with the transducer region interposed in the moving direction.
